(19) United States
(12) Patent Application Publication
(10) Pub. No.: US 2024/0175882 A1
(43) Pub. Date: May 30, 2024

(54) OIL RED O STAINING SOLUTION AND OIL RED O STAINING METHOD

(71) Applicant: Children's Hospital of Chongqing Medical University, Chongqing (CN)

(72) Inventors: Yang BI, Chongqing (CN); Wen JIA, Chongqing (CN); Junbao DU, Chongqing (CN); Yun HE, Chongqing (CN); Li ZHAO, Chongqing (CN)

(21) Appl. No.: 18/431,495

(22) Filed: Feb. 2, 2024

Related U.S. Application Data
(63) Continuation of application No. PCT/CN2023/082270, filed on Mar. 17, 2023.

(30) Foreign Application Priority Data
Jul. 15, 2022 (CN) 202210839471.6

(51) Int. Cl. G01N 33/92; C09B 67/38; G01N 1/30

(52) U.S. Cl. CPC G01N 33/92; C09B 67/008; G01N 1/30; G01N 2001/302; G01N 2001/305

(57) ABSTRACT

The disclosure relates to an improved Oil Red O staining solution, which consists of 0.5% of Oil Red O, 50% of ethanol, and 5% to 10% of salicylic acid. The staining solution does not contain organic solvents toxic to human bodies and has good safety and no toxic effect. The disclosure further relates to an Oil Red O staining method for cells or tissues, which is simple in operation, short in staining time, and easy to stain neutral fat in cells or tissues, with good staining effect and clean background. This method does not use isopropanol, which is safe and convenient and is suitable for popularization and application in laboratories.

### CROSS-REFERENCE TO RELATED APPLICATIONS

This application is a National Stage of International Application No. PCT/CN2023/082270, filed on Mar. 17, 2023, which claims priority to Chinese Patent Application No. 202210839471.6, filed on Jul. 15, 2022. Both of which are hereby incorporated by reference in their entireties.

### TECHNICAL FIELD

The disclosure belongs to cell biology technology, in particular relates to an intracellular lipid staining technology.

### BACKGROUND

Oil Red O staining is a method to show fat in tissues or cells, which is often used in pathological diagnosis and scientific research. Oil Red O is an oil-soluble azo dye, which is a strong lipid solvent and stain, is highly soluble in fat, and can specifically stain neutral lipids such as triglycerides and lipoproteins in cells or tissues. The principle is that solubility of Oil Red O in the neutral lipids is higher than that in staining solutions. During staining, Oil Red O is transferred from a staining solution medium to a tissue fat medium, so that intracellular lipid droplets are stained red. Influencing factors of staining effect of Oil Red O are: 1. Content of Oil Red O in staining solution; 2. Difference in solubility of Oil Red O between a staining medium and a tissue fat medium. The greater the difference, the faster a penetrating velocity in transferring can be, and the more Oil Red O may permeate and collect in tissue fat.

Preparation of an Oil Red O stain solution is very important for staining effect of Oil Red O. Oil Red O is soluble in benzene, chloroform, glacial acetic acid, ether, ethanol, acetone, petroleum ether, non-volatile oil, hot glycerol, and volatile oil, but insoluble in water. Organic solvents are volatile and harmful to human bodies, and precipitation is prone to occur after the stain solution is prepared. In staining, it is easy to leave red crystals on tissue slices, which affects determination of results. These organic solvents themselves can also dissolve fat in tissues, and thus it is necessary to add a certain proportion of water to an original solution to prepare the solvent to reduce its concentration and fat-dissolving ability, prevent the fat in tissues from dissolving, and increase solubility difference of Oil Red O between in the dye medium and the tissue fat medium.

100% of isopropanol saturated Oil Red O solution is convenient to store and is the most conventional commercial reagent, which is prepared to be 60% of isopropanol for staining when used. This method is convenient to operate and has good staining effect and is the most commonly used preparation method of an Oil Red O staining solution at present. However, isopropanol, as an organic solvent, is volatile, and inhalation of isopropanol is harmful to human bodies during operation. Oil Red O has a high solubility in 60% of isopropanol with a deep staining background, and is with poor staining effect for samples with a small number of lipid droplets in cells and a small volume of lipid droplets.

Ethanol is a non-toxic organic solvent, which can be used for Oil Red O staining and is harmless to human bodies. At present, it is reported that 50% of ethanol is used to prepare the Oil Red O staining solution, but solubility of Oil Red O in 50% of ethanol is not high, which presents poor staining effect, light lipid staining and far less straining effect than that of the Oil Red O staining solution prepared by 60% of isopropanol under same conditions.

A molecular formula of salicylic acid is C7H6O3, which is a fat-soluble organic acid and can be dissolved in organic solvents such as ethanol, acetone, and ether. As an important raw material, salicylic acid is often used in fields of fine chemicals such as medicine, perfume, stains, and rubber additives. Salicylic acid has functions of heat-clearing and detoxifying, and inflammation diminishing and sterilizing. Clinically, 2% to 3% of ethanol salicylate (containing 70% of ethanol) is used to inhibit bacteria and produce enzymes to relieve itching. Salicylic acid can also dissolve oil in pores. Clinically, 2% to 10% of ethanol salicylate (including 70% of ethanol) is used to exfoliate, shrink pores, remove blackheads and acne, etc., which has no toxic effect on human bodies.

### SUMMARY

To solve the problems of the toxic effect of an Oil Red O stain solution, deep background of Oil Red O staining and poor staining effect on small oil droplets, an Oil Red O stain solution and an Oil Red O staining method are provided in the disclosure. An object of the disclosure can be achieved by following technical schemes.

An Oil Red O stain solution contains 0.5% of oil red O (by weight), 50% of ethanol (by volume) and 5% to 10% of salicylic acid (by weight).

An Oil Red O staining method for cells is further provided in the disclosure, which includes following steps:
Step 1, inoculating cells to be detected into a culture dish or a culture plate, and adding treatment factors to a detection time point;
Step 2: sucking and removing a culture medium, washing with PBS for 1 to 2 times, and adding 4% of paraformaldehyde and fixing for 20 to 30 minutes;
Step 3: sucking and removing a fixed solution, and washing with PBS for 3 times;
Step 4: preparing the Oil Red O stain solution described above, which is filtered with a filter paper or a 0.45 μm filter for later use;
Step 5: sucking and removing PBS and adding 50% of ethanol for 15 to 20 seconds;
Step 6: sucking and removing 50% of ethanol, adding the Oil Red O stain solution obtained in Step 4, and incubating at room temperature for 10 to 15 min;
Step 7: sucking and removing the Oil Red O stain solution, and adding 50% of ethanol for differentiation for 15 to 20 seconds, and washing with PBS 3 times until liquid is clear, and then photographing under a microscope; and
Step 8: sucking and removing PBS, adding 100% of ethanol to dissolve Oil Red O, adding 50 μl of ethanol solution into a 96-well plate, and detecting an OD value at a wavelength of 492 nm by an enzyme-linked analyzer.

Preferably, the cells are mammalian cells containing neutral lipids.

An Oil Red O staining method for tissues is further provided in the disclosure, which includes following steps:
Step 1: preparing tissue containing neutral lipid (liver, kidney, fat, aorta) into frozen slices with a thickness of 12 μm immediately after sampling;
Step 2: fixing the slices with 4% of paraformaldehyde for 10 minutes, washing with distilled water for 5 minutes, and soaking with 70% of ethanol for 15 to 20 seconds;
Step 3: preparing the Oil Red O stain solution described above, which is filtered with a filter paper or a 0.45 μm filter for later use;
Step 4: dip staining with the Oil Red O stain solution for 10 to 15 min;
Step 5: differentiating with 70% of ethanol until stroma is clear, and washing with distilled water for 5 minutes;
Step 6: counterstaining with hematoxylin for 3 to 5 minutes and washing with distilled water for 5 minutes; and
Step 7: sealing the slices with glycerol gelatin and observing under a microscope.

### Technical Effect:

In the disclosure, a salicylic acid ethanol solution is adopted to prepare Oil Red O, which has no toxic effect on human bodies,

The Oil Red O staining method according to the disclosure is convenient to operate, with short staining time and clean staining background, and the staining effect of the Oil Red O staining solution prepared by using 50% of ethanol and 5 to 10% of salicylic acid as a solvent is equivalent to that of the Oil Red O staining solution prepared by 60% of isopropanol, even better than that of the Oil Red O staining solution prepared by 60% of isopropanol.

The Oil Red O solution prepared with a salicylic acid-ethanol solution can be stored for a long time (more than half a year), which facilitates making an Oil Red O staining kit.

### BRIEF DESCRIPTION OF DRAWINGS

FIG. 1 shows Oil Red O stain prepared with different solvents:
(1) 100% of isopropanol; (2) 60% of isopropanol; (3) 100% of ethanol; (4) 70% of ethanol; (5) 50% of ethanol; (6) 50% of ethanol+1% of salicylic acid; (7) 50% of ethanol+2% of salicylic acid; (8) 50% of ethanol+5% of salicylic acid; (9) 50% of ethanol+10% of salicylic acid; (10) 30% of ethanol; (11) 30% of ethanol+1% of salicylic acid; (12) 30% of ethanol+2% of salicylic acid; (13) 30% of ethanol+5% of salicylic acid; (14) 30% of ethanol+10% of salicylic acid; (15) 70% of ethanol+10% of salicylic acid;

FIG. 2 shows OD values of Oil Red O stain solutions prepared with different solvents at a wavelength of 492 nm by the enzyme-linked analyzer;

FIG. 3 shows staining of 143B human osteosarcoma cells with Oil Red O stains prepared with different solvents (400×);

FIG. 4 shows the OD values of Oil Red O in cells of FIG. 3 after elution at the wavelength of 492 nm by the enzyme-linked analyzer;

FIG. 5 shows the staining of HSC-T6 rat hepatic stellate cells with Oil Red O stains prepared with different solvents (400×);

FIG. 6 shows the OD values of Oil Red O in cells of FIG. 5 after elution at the wavelength of 492 nm by the enzyme-linked analyzer;

FIG. 7 shows the staining of APRE-19 human retinal epithelial cells with Oil Red O stains prepared with different solvents (200×);

FIG. 8 shows the OD values of Oil Red O in cells of FIG. 7 after elution at the wavelength of 492 nm by the enzyme-linked analyzer;

FIG. 9 shows the staining of adipocytes with Oil Red O stains prepared with different solvents (200×);

FIG. 10 shows the OD values of Oil Red O in cells of FIG. 9 after elution at the wavelength of 492 nm by the enzyme-linked analyzer;

FIG. 11 shows the staining of liver, aorta, skin, and adipose tissue slices of rat with Oil Red O stains prepared by different solvents (100×): (2) 60% of isopropanol; (5) 50% ethanol; (8) 50% of ethanol+5% of salicylic acid; (9) 50% of ethanol+10% of salicylic acid.

FIG. 12 shows quantitative comparison of the staining effect of groups (2), (5), (8), and (9) for the liver, aortic arch, skin, and adipose tissue s.

### DESCRIPTION OF EMBODIMENTS

In the following, the present disclosure will be described in detail in the examples. It is necessary to point out that the following embodiments are only used to further illustrate the present disclosure, and cannot be understood as limiting the scope of protection of the present disclosure. Those skilled in the art can make some non-essential improvements and adjustments to the present disclosure based on above summary.

### Example 1. Preparation of Oil Red O Stain Solution

An Oil Red O stain solution with 60% isopropanol was prepared with 0.50 saturated Oil Red O and ddH2O in a ratio of 3:2, as a control group.

2) Several 50 ml centrifugal tubes were prepared, and 0.2 g of Oil Red O powder was added to each tube (if there is any deviation in weighing, 100 ml of solvent was added per 0.5 g of Oil Red O to prepare a 0.5% Oil Red O solution). 20 g of salicylic acid was weighed and dissolved in 60 ml of ethanol and 40 ml of ddH2 to prepare a 20% salicylic acid-ethanol (containing 60% ethanol) solution.

Solvents were prepared according to the following table (per 10 ml):

| | 100% of | 20% of salicylic | |
|---|---|---|---|
| | ethanol | acid and 60% | ddH₂O |
| Solvent | [symbol] | of ethanol (ml) | (ml) |
| 70% of ethanol | 7 | / | 3 |
| 50% of ethanol | 5 | / | 5 |
| 50% of ethanol + 1% of [symbol] | 4.7 | 0.5 | 4.8 |
| 50% of ethanol + 2% of [symbol] | 4.4 | 1 | 4.6 |
| 50% of ethanol + 5% of [symbol] | 3.5 | 2.5 | 4 |
| 50% of ethanol + 10% | 2 | 5 | 3 |
| 30% of ethanol | 3 | / | 7 |
| 30% of ethanol + 1% of [symbol] | 2.7 | 0.5 | 6.8 |
| 30% of ethanol + 2% of [symbol] | 2.4 | 1 | 6.6 |
| 30% of ethanol + 5% of [symbol] | 1.5 | 2.5 | 6.0 |
| 30% of ethanol + 10% | 1 | 5 | 5 |

[symbol]

indicates data missing or illegible when filed

40 ml of the above solvents were added to a centrifuge tube to prepare a 0.50% Oil Red O solution, blown evenly, and stood for 10 minutes. Dissolution of Oil Red O is shown in FIG. 1, and an order of solubility from large to small observed with eyes is as follows:
(1)=(3)=(15)>(9)=(2)>(8)>(4)>(7)=(14)>(6)>(5)>(13)>(12)>(11)>(10).

At the same ethanol concentration, the solubility of Oil Red O increases with increase in a salicylic acid concentration. Groups with 30% of ethanol+5% of salicylic acid and 30% of ethanol+10% of salicylic acid groups present flocculent precipitation, which is undissolved salicylic acid. Ethanol can be added to a concentration above 50% to dissolve the flocculent precipitation.

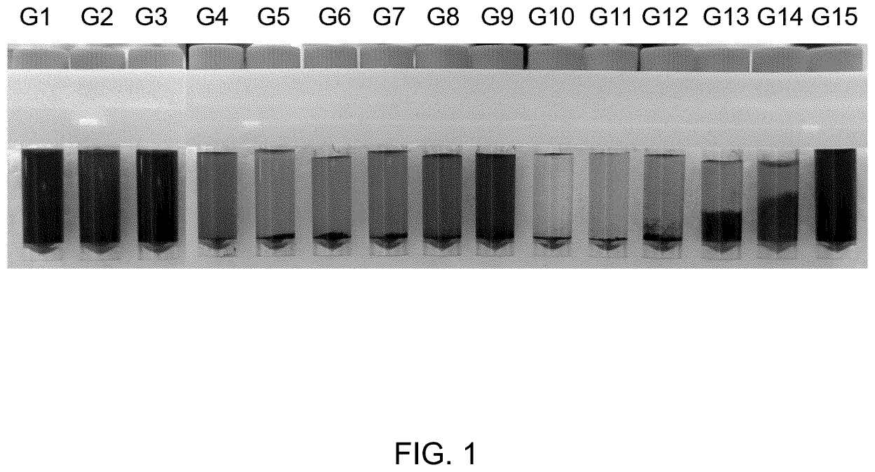

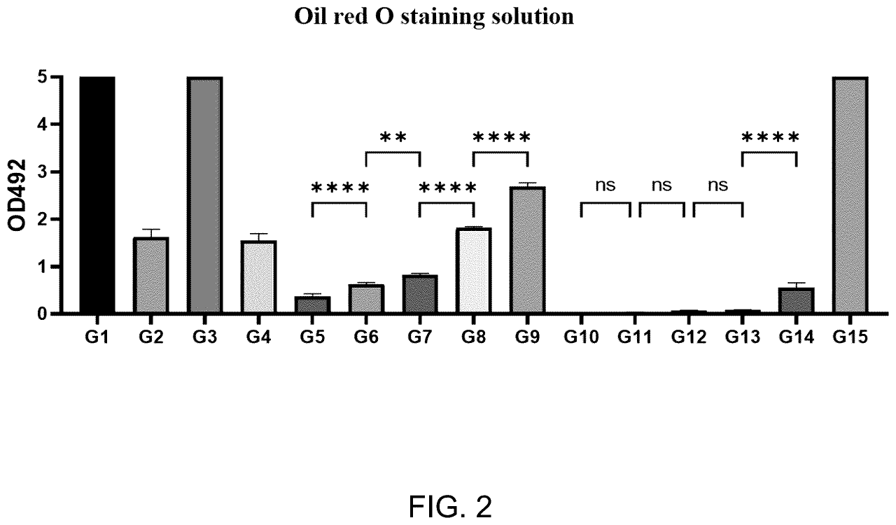

The above Oil Red O solution was filtered with a filter paper or a 0.45 μm filter, 50 μl of the solution was taken and put into a 96-well plate, and OD values were measured at a wavelength of 492 nm with the enzyme-linked analyzer. Results (Table 1 and FIG. 2) are as follows:

| Groups | Group 1 | Group 2 | Group 3 | Group 4 | Group 5 |
|---|---|---|---|---|---|
| OD492 | OVERFLOW | 1.625 ± 0.226 | OVERFLOW | 1.5645 ± 0.203 | 0.367 ± 0.061 |
| Groups | Group 6 | Group 7 | Group 8 | Group 9 | Group 10 |
| OD492 | [symbol] 635 ± 0.046 | 0.832 ± 0.037 | 1.821 ± 0.039 | 2.679 ± 0.107 | 0.021 ± 0.002 |
| Groups | Group 11 | Group 12 | Group 13 | Group 14 | Group 15 |
| OD492 | [symbol] 37 ± 0.001 | 0.075 ± 0.001 | 0.083 ± 0.009 | 0.569 ± 0.138 | OVERFLOW |

[symbol]

indicates data missing or illegible when filed

### Example 2. Compare Staining of 143B Human Osteosarcoma Cells with Oil Red O Staining Solutions Prepared with Different Solvents

Cell preparation: 143B human osteosarcoma cells were inoculated into a 48-well plate, adhered for 24 hours and then stained.

Cell fixation: a culture medium was sucked and removed, 500 μl of PBS was added to each well for washing 1 to 2 times, and 4% of paraformaldehyde was added to fix for 20 to 30 minutes, and fixing liquid was sucked and removed and 500 μl of PBS was added to each well for washing for 3 times.

Oil Red O staining: PBS in the well plate was sucked and removed, 50% of ethanol of 500 V was added to wash for 15 to 20 seconds to remove water; 200 μl of different Oil Red O stains filtered in Example 1 were added into the wells respectively, which was incubated at a room temperature for 10 to 15 minutes; the Oil Red O stain solution was sucked and removed, 50% of ethanol of 500 V was added to wash for 15 to 20 seconds, and differentiation was performed to remove excess stain solution; and washing was performed with PBS for more than 3 times until liquid was clear, and photographs were made under a microscope.

The above staining was performed in triplicate, in which group (1) and group (2) were washed and differentiated with 60% isopropanol.

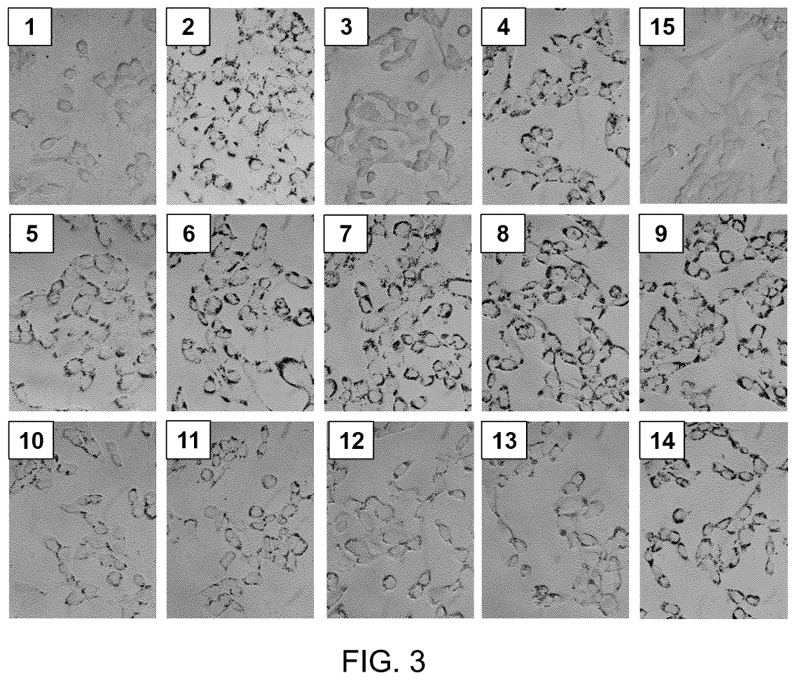

Groups with 30% ethanol+5% salicylic acid and 30% ethanol+10% salicylic acid groups present flocculent precipitation, which is undissolved salicylic acid. Flocculent precipitation can be removed by centrifugation or filtration without affecting the staining results of Oil Red O. Ethanol can be added to a concentration above 50% to dissolve the flocculent precipitation. The Oil Red O stain solution with 70% ethanol+10% salicylic acid and 100% ethanol as a solvent has high solubility for Oil Red O with a red and bright color, which is equivalent to that of a 100% isopropanol solution, but with poor staining effect, and lipid in cells can hardly be stained, and under same washing conditions, the background is still light red (FIG. 3: Group 1, Group 3 and Group 5).

Except for Groups 1, 3, and 15, Oil Red O stain solutions of other groups can stain lipid droplets. Lipid droplets in Groups 10, 11, 12, and 13 were pale yellow; and lipid droplets in other groups were orange to deep red. Red staining of lipid droplets in the cytoplasm can be seen in Groups (8) and (9) in 5 minutes, and the staining effect of lipid droplets is best when staining for 10 to 15 minutes, which is no different from that of lipid droplets stained for more than 2 hours or even overnight (12 to 16 hours), with clean staining background and no impurities and red crystals.

The staining effect of Oil Red O under a microscope is shown in FIG. 3, the staining area and colors of lipid droplets from deep to shallow are as follows:
(2)=(9)=(8)>(7)>(6)=(14)=(4)>(5)>(13)>(12)>(11)>(10)>(1)=(3)=(15).

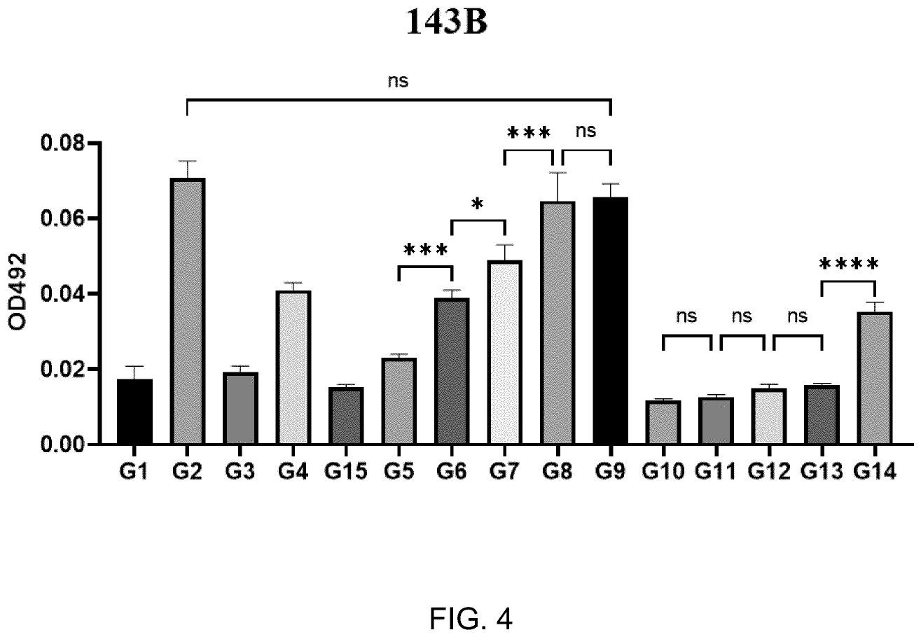

PBS was sucked and removed (it can be chosen to wipe with a cotton swab dipped in 50% of ethanol to remove excessive Oil Red O stain solution on side walls of small holes of the culture plate), 100% of ethanol of 100 μl was added, incubated at the room temperature for 10 minutes, to dissolve the Oil Red O in the lipid droplets, 50 μl of ethanol solution was sucked into a 96-well plate, and the 96-well plate is place at a wavelength of 492 nm to measure OD values (FIG. 4).

To sum up, Group (9) with 50% of ethanol+10% of salicylic acid and Group (8) with 50% of ethanol+5% of salicylic acid have the best staining effect, which is equivalent to Group (2) with 60% of isopropanol, and small lipid droplets in Groups (9) and (8) are more clearly stained than that in Group (2), followed by Group (7) with 50% of ethanol+2% of salicylic acid.

### Example 3. Compare Staining of HSC-T6, APRE-19 Cells and Adipocytes with Oil Red O Staining Solutions Prepared with Different Solvents

HSC-T6 rat hepatic stellate cells were inoculated into a 48-well plate with an initial cell density of 15 to 20%. After the cells were adhered, they were treated with a DMEM complete medium containing 100 μmol/L of sodium oleate and 10 μmol/L of all-trans retinoic acid for 5 to 7 days.

APRE-19 human retinal epithelial cells were inoculated into a 48-well plate with an initial cell density of 20 to 30%. After the cells were adhered, they were treated with DMEM/F12 complete medium containing 100 μmol/L of sodium oleate for 48 hours.

Primary adipocytes were extracted from fat tissue of 5-week-old rat and cultured for three days by using a modified ceiling culture method, slides with adherent adipocytes were put in a 12-well plate and fixed for staining.

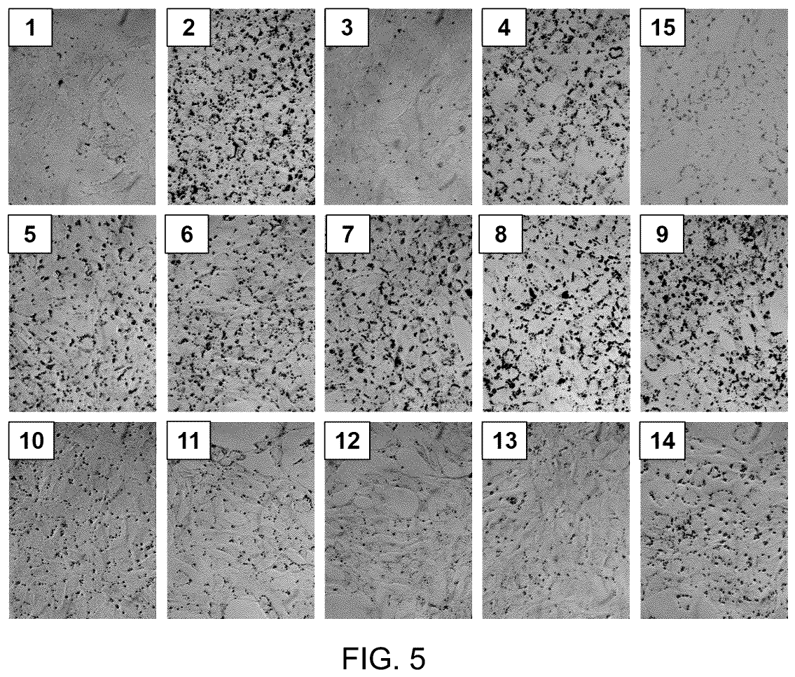

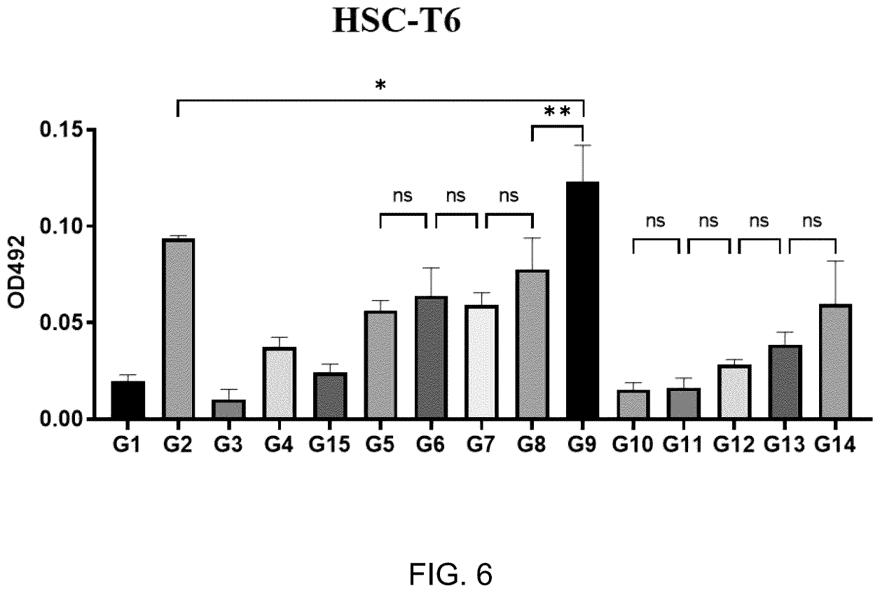

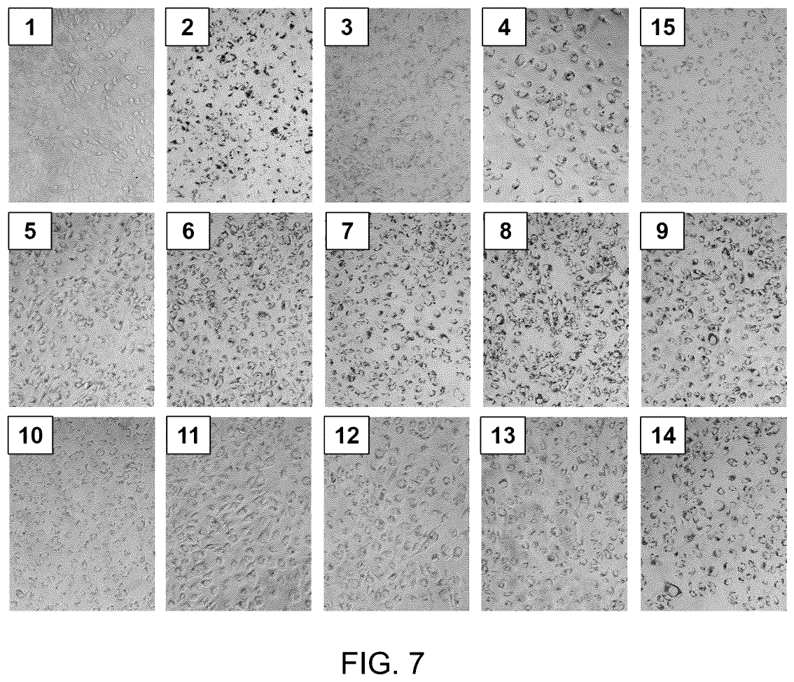

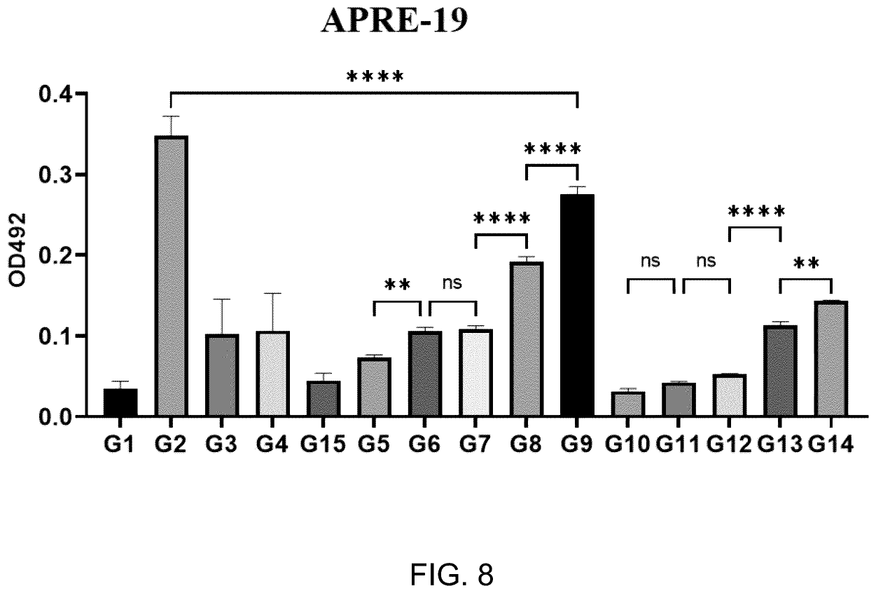

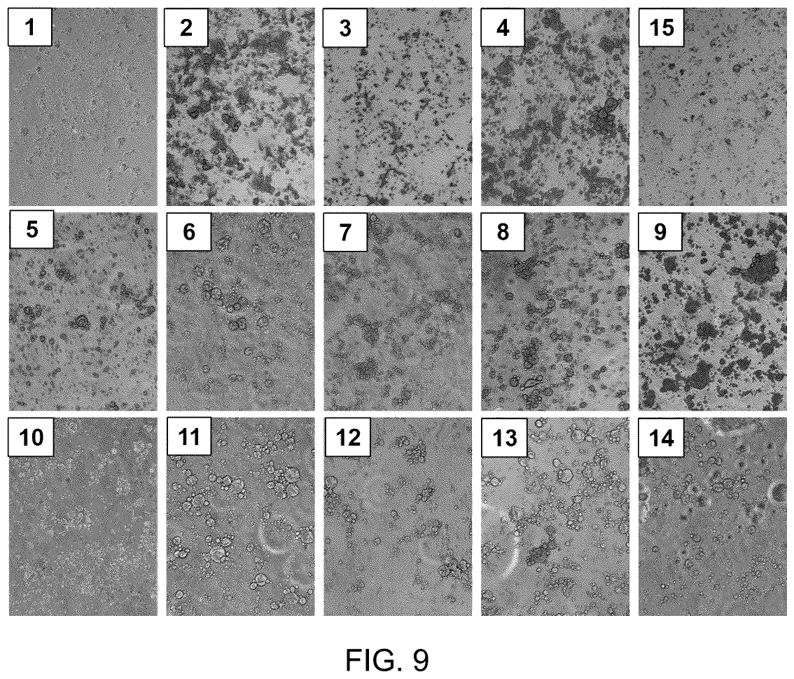

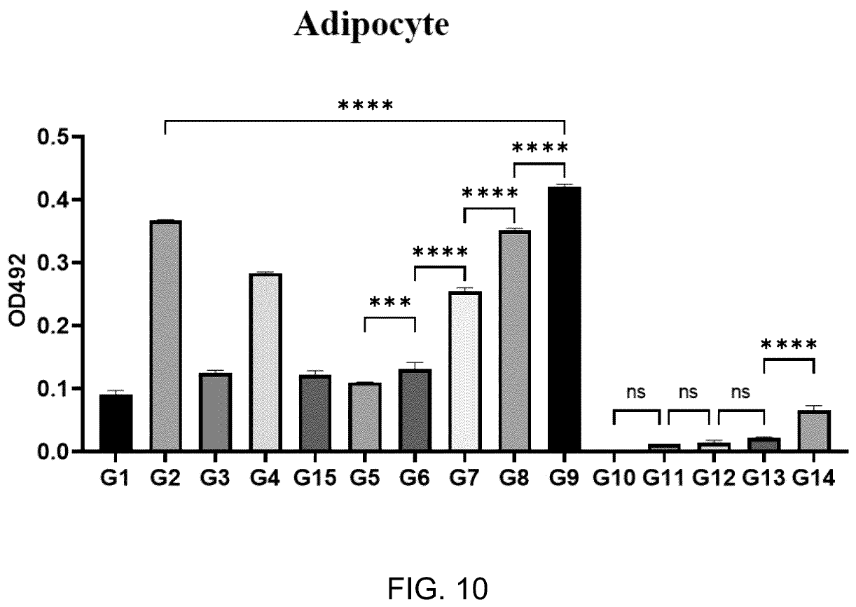

The above Oil Red O staining solutions are used for staining, and staining results of HSC-T6 rat hepatic stellate cells are shown in FIG. 5 and FIG. 6. Staining results of APRE-19 human retinal epithelial cells are shown in FIG. 7 and FIG. 8. Staining results of adipocytes are shown in FIG. 9 and FIG. 10. A trend of staining effect Oil Red O staining solutions prepared with different solvents is the same as that of 143B human osteosarcoma cells, and Group (9) with 50% ethanol+10% salicylic acid and Group (8) with 50% ethanol+5% salicylic acid have best Oil Red O staining effect, which is equivalent to even exceeds Group (2) with 60% isopropanol, followed by Group (7) with 50% ethanol+2% salicylic acid.

In addition to the above cells, other mammalian cells containing neutral lipids can be stained with Oil Red O using this method.

### Example 4. Staining Effect of Improved Oil Red O Staining Solution on Tissue Samples Containing Neutral Lipids

6-8 weeks old SD rats were fed with a 60% high-fat diet for at least 2 weeks and then euthanized humanely using CO2 by inhalation (100% CO2) followed by cervical dislocation. Next, the liver, aorta, skin, and adipose tissue were collected and rapidly frozen in liquid nitrogen-cooled isopentane and embedded in Tissue-Tek. Sections of each tissue sample were cut to a thickness of 12 μm.

Then the slices were fixed with 4% paraformaldehyde for 10 minutes, fully washed with distilled water for 5 minutes to remove formaldehyde, bathed in 70% ethanol (the control group was bathed with 60% isopropanol), dip stained with an Oil Red O staining solution (Oil Red O staining solutions prepared with 50% ethanol+10% salicylic acid and 50% ethanol+5% salicylic acid were selected, and an Oil Red O stain solution prepared with 60% of isopropanol as control) for 10 to 15 minutes, differentiated with 70% of ethanol until stroma was clear (the control group was differentiated with 60% of isopropanol), fully washed with distilled water for 5 minutes, counterstained with hematoxylin for 3 to 5 minutes, and washed with distilled water for 5 minutes, sealed with glycerol gelatin and observed under a microscope.

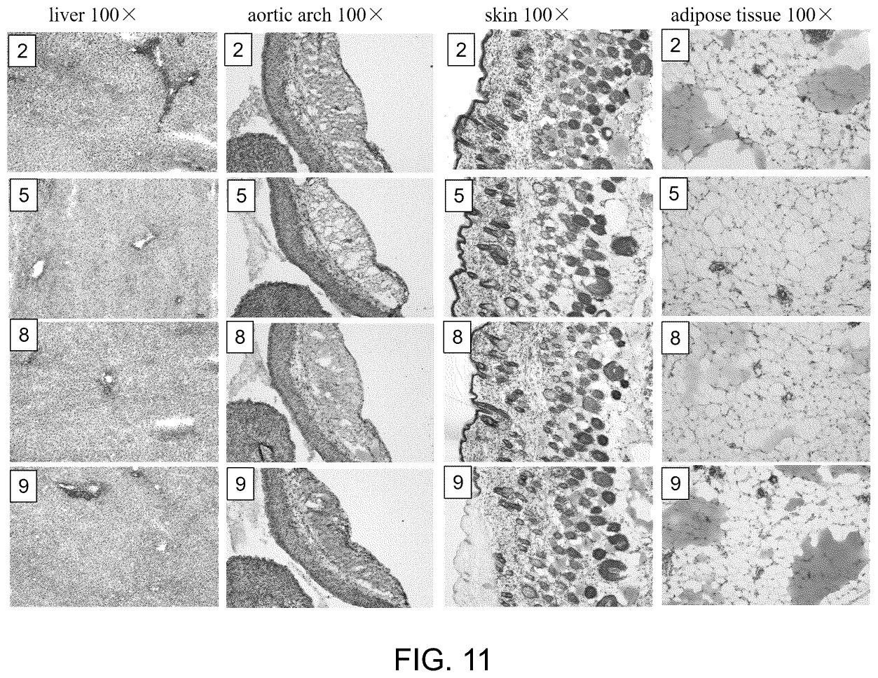

The staining effect of Oil Red O is shown in FIG. 11. The tissues stained by Group (9) with 50% of ethanol+10% of salicylic acid and Group (8) with 50% of ethanol+5% of salicylic acid present clean background, no red crystals and impurities, with lipid droplets being evenly distributed in the cytoplasm of hepatocytes and a clear nuclear staining. In Group (2) with 60% of isopropanol, little purple-red to red-black impurities and red fat floating on the nucleus can be seen, and a light-yellow stain was observed in Group (5) with 50% ethanol. Lipid droplets in Group (9) with 50% ethanol+10% of salicylic acid and Group (2) with 60% of isopropanol are dark red, while those in Group (8) with 50% of ethanol+5% of salicylic acid are orange-red, and there are more small red lipid droplets in hepatocytes in Group (9) with 50% of ethanol+10% of salicylic acid than in Group (2) with 60% of isopropanol.

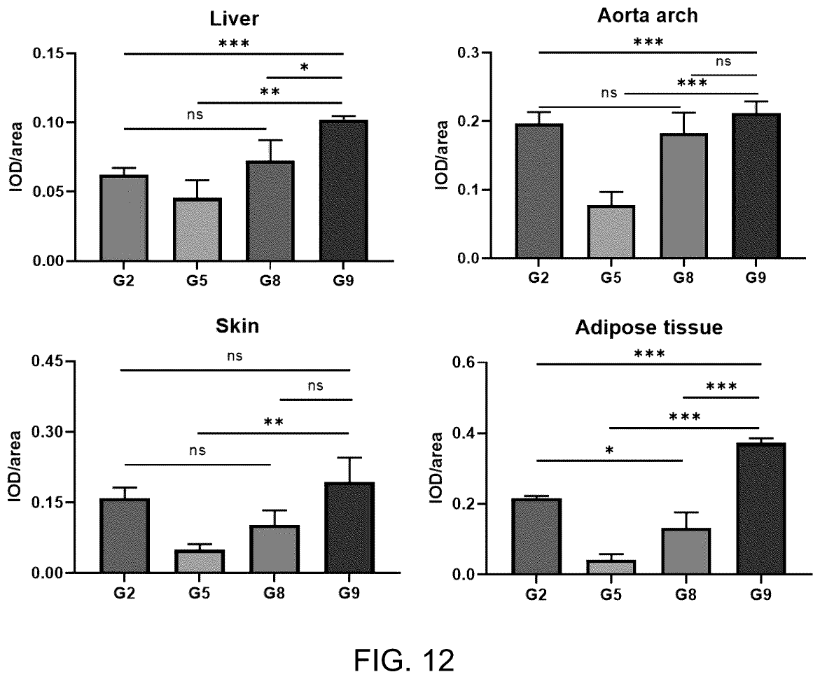

Then a quantitative comparison of the staining effect was performed by Image-Pro Plus software, and the results showed in FIG. 12 were consistent with the trend under the light microscope.

In addition to the liver, aorta, skin, and adipose tissue, other tissues containing neutral lipids can be stained using this method.

What is claimed is:

1. An Oil Red O stain solution, comprising 0.5% of Oil Red O by weight, 50% of ethanol by volume, and 5% to 10% of salicylic acid by weight.

2. An Oil Red O staining method for cells, comprising:
Step 1, inoculating cells to be detected into a culture dish or a culture plate, and adding treatment factors to a detection time point;
Step 2: sucking and removing a culture medium, washing with PBS for 1 to 2 times, and adding 4% of paraformaldehyde and fixing for 20 to 30 minutes;
Step 3: sucking and removing a fixed solution, and washing with PBS for 3 times;
Step 4: preparing the Oil Red O stain solution according to claim 1, which is filtered with a filter paper or a 0.45 μm filter for later use;
Step 5: sucking and removing PBS and adding 50% of ethanol for 15 to 20 seconds;
Step 6: sucking and removing 70% of ethanol, adding the Oil Red O stain solution obtained in Step 4, and incubating at room temperature for 10 to 15 min;
Step 7: sucking and removing the Oil Red O stain solution, and adding 50% of ethanol for differentiation for 15 to 20 seconds, and washing with PBS 3 times until liquid is clear, and then photographing under a microscope; and
Step 8: sucking and removing PBS, adding 100% of ethanol to dissolve Oil Red O, adding 50 μl of ethanol solution into a 96-well plate, and detecting an OD value at a wavelength of 492 nm by an enzyme-linked analyzer.

3. The staining method according to claim 2, wherein the cells are mammalian cells containing neutral lipids.

4. An Oil Red O staining method for tissues, comprising:
Step 1: preparing tissue containing neutral lipid into frozen slices with a thickness of 12 μm immediately after sampling;
Step 2: fixing the slices with 4% of paraformaldehyde for 10 minutes, washing with distilled water for 5 minutes, and soaking with 70% of ethanol for 15 to 20 seconds;
Step 3: preparing the Oil Red O stain solution according to claim 1, which is filtered with a filter paper or a 0.45 μm filter for later use;
Step 4: dip staining with the Oil Red O stain solution for 10 to 15 min;
Step 5: differentiating with 70% of ethanol until stroma is clear, and washing with distilled water for 5 minutes;
Step 6: counterstaining with hematoxylin for 3 to 5 minutes and washing with distilled water for 5 minutes; and
Step 7: sealing the slices with glycerol gelatin and observing under a microscope.
